(19) United States
(12) Patent Application Publication
(10) Pub. No.: US 2015/0329890 A9
(43) Pub. Date: Nov. 19, 2015

(54) ERROR CORRECTION IN NUCLEIC ACID MOLECULES

(71) Applicant: Jingdong TIAN, Chapel Hill, NC (US)

(72) Inventor: Jingdong TIAN, Chapel Hill, NC (US)

(21) Appl. No.: 13/864,093

(22) Filed: Apr. 16, 2013

Related U.S. Application Data
(60) Provisional application No. 61/624,708, filed on Apr. 16, 2012.

(51) Int. Cl. C12P 19/34

(52) U.S. Cl. CPC C12P 19/34

(57) ABSTRACT

An enzymatic method for removing sequence errors in nucleic acid molecules are described. The method utilizes a CEL endonuclease that cuts heteroduplexes at mismatch sites containing the errors and an overlap extension polymerase chain reaction to re-assemble the cleaved fragments into full-length nucleic acid molecules free of the errors.

### CROSS-REFERENCE TO RELATED APPLICATION

This application is entitled to priority pursuant to 35 U.S.C. §119(e) to U.S. Provisional Patent Application No. 61/624708, filed Apr. 16, 2012, the content of which is hereby incorporated by reference in its entirety.

### FIELD OF THE INVENTION

This invention generally relates to nucleic acid synthesis. In particular, this invention relates to the correction of sequence errors within nucleic acid molecules.

### BACKGROUND

Gene and genome syntheses are playing an increasingly important role in synthetic biology and biotechnology. To increase throughput and reduce cost, new gene synthesis methods that take advantage of DNA microarrays and microfluidic devices have recently been demonstrated. However, each coupling step has an intrinsic error rate. Removing errors that arise from oligonucleotide (oligo) synthesis and gene assembly remains a significant challenge, especially for gene synthesis using microarray-produced oligonucleotides, where error rates tend to be higher (Tian et al, Nature 432:1050-1054 (2004), Borovkov et al, Nucleic Acids Res. 38:s180 Epub (2010)).

Errors in gene synthesis are typically controlled in two ways: 1) the individual oligonucleotides can each be purified to remove error sequences; 2) the final assembled products are sequenced to discover if errors are present. To improve the quality of oligonucleotides used in construction of longer nucleic acid molecules, size exclusion purification using polyacrylamide gel electrophoresis (PAGE) (Ellington and Pollard, Jr., Curr. Protoc. Nucleic Acid Chern, Appendix 3, Appendix 3C), or high performance liquid chromatography (HPLC) (Andrus and Kuimelis, Curr. Protoc. Nucleic Acid Chern, Chapter 10, Unit 10 15) can be used to remove oligonucleotides that can result in large insertions and deletions. An array hybridization method has also been developed to reduce errors in chip-generated oligo pools, which requires special microarrays of complementary oligonucleotides (Tian et al, Nature 432:1050-1054 (2004)). Using next-generation sequencing technology, it may also be feasible to sequence and select correct oligo sequences for gene construction, as a recent proof-of-concept experiment has demonstrated (Matzas et al., Nat. Biotechnol. 2010; 28:1291-1294).

To eliminate errors in longer synthetic gene constructs, slow and labor-intensive cloning and sequencing methods are traditionally used. If the error rate is high or the sequence is long, large numbers of clones need to be sequenced in order to identify a correct sequence (Carr et al., Nucleic Acids Res. 2004; 32:e162). If a perfect clone cannot be isolated, site-directed mutagenesis needs to be used to fix errors identified by sequencing (Heckman et al., Nat. Protoc. 2007; 2:924-932; Rabhi et al., Mol. Biotechnol. 2004; 26:27-34; Xiong et al., Nat. Protoc. 2006; 1:791-797; Linshiz et al., Mol. Syst. Biol. 2008; 4:191; Marsic et al., BMC Biotechnol. 2008; 8:44). Multiple rounds of cloning, sequencing and site-directed mutagenesis can significantly increase the cost and turn around time for gene synthesis.

In order to increase the chance of finding a correct clone, the overall error frequency in the synthetic gene pool needs to be significantly reduced. Methods of using mismatch-binding proteins (e.g. MutS) to remove error-containing DNA heteroduplexes have been developed (Carr et al., Nucleic Acids Res. 2004; 32:e162; Smith et al., Proc. Natl Acad. Sci. USA. 1997; 94:6847-6850; Binkowski et al., Nucleic Acids Res. 2005; 33:e55). However, MutS-based methods theoretically do not work well for error-rich sequences, because the correct sequences have to outnumber the erroneous sequences in order to avoid being depleted from the synthetic pool.

Enzymatic mismatch cleavage represents another option for the removal of mismatched bases from synthetic genes. Mismatch-cleaving enzymes can cleave heteroduplexes at the vicinity of the mismatch sites, which allows the mutant bases to be subsequently removed by exonuclease activity present in the reaction mixture. A number of enzymes have been tested, including T7 endonuclease I, T4 endonuclease VII and Escherichia coli endonuclease V, which showed various effectiveness due to various specificities of the enzymes (Young et al., Nucleic Acids Res. 2004; 32:e59; Fuhrmann et al., Nucleic Acids Res. 2005; 33:e58; Bang et al., Nat. Methods. 2008; 5:37-39). For example, T7 endonuclease I showed no detectable activity with model mismatch substrates tested, substrate-dependent efficiency of second-strand cleavage was observed for Escherichia coli endonuclease V (Fuhrmann et al., Nucleic Acids Res. 2005).

Therefore, there exists a need for an improved method and composition for correcting errors in nucleic acid molecules. Embodiments of the present application relate to such method and composition.

### BRIEF SUMMARY OF THE INVENTION

The present invention relates generally to removing sequence errors in a nucleic acid molecule, such as a gene.

In one general aspect, the present invention provides a method for removing one or more errors in a plurality of nucleic acid molecules. The method generally comprises treating heteroduplexes comprising one or more mismatch sites resulting from one or more sequence errors with a CEL endonuclease to cleave the heteroduplexes at the mismatch site(s). The cleaved heteroduplexes are then corrected and re-assembled into intact, error-free nucleic acid molecules by performing an overlap-extension polymerase chain reaction amplification using a DNA polymerase having 3′-5′ exonuclease activity. A method for removing one or more errors in a plurality of nucleic acid molecules according to the invention can be used to remove errors such as nucleotide substitutions, deletions and insertions of 1-12 nucleotides in the sequence.

According to embodiments of the present invention, a method for removing one or more sequence errors in a plurality of nucleic acid molecules comprises:
(1) heating and subsequently cooling the plurality of nucleic acid molecules, thereby forming one or more heteroduplexes, wherein the heteroduplex comprises one or more mismatch sites resulting from the sequence errors;
(2) contacting the one or more heteroduplexes with a CEL endonuclease under conditions for effective cleavage of the one or more heteroduplexes at the one or more mismatch sites, thereby obtaining cleaved fragments; and
(3) contacting the cleaved fragments with a DNA polymerase having 3′-5′ exonuclease activity under conditions for an overlap extension polymerase chain reaction amplification, thereby producing a plurality of nucleic acid molecules free of the one or more errors.

In a preferred embodiment, the CEL endonuclease is a CEL II endonuclease from celery. In yet another preferred embodiment, the DNA polymerase having 3′-5′ exonuclease activity is Phusion polymerase. In yet another embodiment, the heteroduplexes are incubated with CEL II endonuclease from celery and a DNA ligase at 35-55° C. for 10-60 min for effective cleavage of the one or more heteroduplexes at the one or more mismatch sites, thereby obtaining cleaved fragments of the heteroduplexes.

In another general aspect, the present invention provides a kit for removing one or more sequence errors in a plurality of nucleic acid molecules. According to embodiments of the present invention, a kit comprises:
(1) a CEL endonuclease, a DNA ligase and a buffer solution for cleaving a heteroduplexe at one or more mismatch sites;
(2) a DNA polymerase having 3′-5′ exonuclease activity and reagents for performing an overlap extension polymerase chain reaction amplification; and
(3) instructions on using the kit for removing one or more sequence errors in a plurality of nucleic acid molecules.

In order for the aspects of the present invention to be more clearly understood, various embodiments will be further described in the following detailed description of the invention with reference to the accompanying drawings. The drawings and following detailed description are intended to provide examples of various embodiments of the present invention. It should be understood that the scope of the invention is not limited by the drawings and discussion of these specific embodiments.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

The patent or application file contains at least one drawing executed in color. Copies of this patent or patent application publication with color drawings will be provided by the Office upon request and payment of the necessary fee.

In the drawings:

FIG. 1 is a schematic representation of the steps involved in a method of removing one or more errors in a nucleic acid molecule according to the invention Nucleic acid molecules are heat denatured and then slowly cooled down to form heteroduplexes containing mismatches at the error sites (left panel); heteroduplexes are cleaved by a CEL endonuclease, such as Surveyor nuclease, at the sites flanking the mismatch bulges; the resulting single-stranded overhangs, where mismatch bases are located, are removed by the proofreading 3′→5′ exonuclease activity of the DNA polymerase, such as Phusion polymerase, used in the overlap-extension PCR (OE-PCR); the resulting fragments with mismatch bases removed are efficiently assembled back into full-length gene constructs during OE-PCR (right panel);

FIG. 2 is an image of an agarose gel showing the cleavage and reassembly of cleaved heteroduplexes during an error correction reaction (ECR) according to a method of the present invention: synthetic rfp gene (lane 1) was incubated with Surveyor nuclease for 20 min (lane 2) and 60 min (lane 3) at 42° C.; the cleavage reaction products (lane 2 and 3) were then re-assembled by OE-PCR into full-length gene products (lane 4 and 5, respectively, marked by arrow); the reaction products were analyzed by agarose gel electrophoresis (lane “M” is a DNA molecular weight marker);

FIGS. 3A and 3B are graphical representations of the results of an ECR performed according to an embodiment of the present invention, as measured by gene function or reporter assays; percentage of functional or fluorescent clones was measured before and after one or two iterations of ECR for 5 different gene constructs; FIG. 3A show the effects of Surveyor incubation time (20 and 60 min) and number of ECR iterations on the synthesis of an rfp gene with the correct sequence by counting fluorescent colonies; FIG. 3B shows the percentage of blue (lacZa—v1&2) or fluorescent colonies (constructs—3&4) after 1 or 2 ECR iterations;

FIG. 4 is an agarose gel image showing RFP gene product that underwent ECR (incubated with Surveyor nuclease) followed by OE-PCR amplification according to the invention, the amounts of nuclease and enhancer in Surveyor incubations were varied as follows: Lanes 1 and 4 (0.5 μl each), lanes 2 and 5 (1 μl each), and lanes 3 and 6 (2 μl each); the incubation temperature was also varied as follows: Lanes 1-3 (42° for 20 min) and lane 4-6 (25° for 60 min); results from all lanes appear similar, with no noticeable difference with increased enzyme amounts;

FIG. 5 show fluorescent images of agarose gel plates with colonies expressing synthesized RFP gene that underwent error correction according to a method of the invention: employing error correction increases the percentage of fluorescent RFP colonies; images are examples of the increased fluorescent population derived by employing ECR with Surveyor nuclease; Panels A1 and A show colonies derived from the uncorrected RFP synthesis product that only yields 50.2% fluorescing colonies; iterations of correction, using 20 min incubations in this case, yield colonies with an increased fluorescent population as shown in Panels B1 and B (first iteration) and Panels C1 and C (second iteration) Panels A1, B1 and C1 are the same images as Panels A, B, and C with an added pseudo-colored red mask to highlight the brightly fluorescent colonies;

FIGS. 6A and 6B are graphs showing the predicted effects of ECR as a function of sequence length; FIG. 6 A shows that the purity of gene synthesis products (as a percentage of error-free clones) decreases exponentially with the length of the product synthesized; employing ECR (1 error in 8,701 bp, blue line) dramatically increases the probability of locating an error-free clone as compared to locating an error-free clone in an uncorrected population (1 error in 526 bp, red line); FIG. 6B shows that employing ECR significantly reduces the number of colonies that need to be screened to have a high (95%) probability of obtaining at least one error-free clone; two iterations of 60 min cleavage incubations with Surveyor (blue line) could yield a correct 10 kb product by sequencing 8 random clones; plots are derived from the result of model calculations as described; and

FIGS. 7A and 7B are graphical representations of the statistical evaluation of errors in synthetic RFP genes with and without Surveyor nuclease treatment: FIG. 7A is a bar graph showing the percentage of fluorescent RFP colonies with and without error correction using Surveyor nuclease; on-chip RFP gene synthesis without error correction resulted in 50.2% fluorescent colonies while those treated with the Surveyor nuclease yielded an 84% fluorescent population; the total number of colonies in each population was approximately 3,000; FIG. 7B is a graph showing the predicted correlation of the probability for an error-free clone as a function of product length (Fuhrmann et al, Nucl. Acids. Res. 33:e58 (2005)) before and after error correction with Surveyor nuclease; error correction using Surveyor nuclease (error frequency f_{c}=0.19 per kb, blue line) increases the probability of locating an error-free clone as compared to locating an error free clone among the uncorrected population (error frequency fᵤ=1.9 per kb, red line), thereby drastically reducing the number of colonies that need to be screened; the error frequencies were calculated from sequencing data in Table 2.

### DETAILED DESCRIPTION OF THE INVENTION

Unless defined otherwise, all technical and scientific terms used herein have the same meaning as is commonly understood by one of ordinary skill in the art to which this invention pertains. All publications and patents referred to herein are incorporated by reference. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

It must be noted that as used herein and in the appended claims, the singular forms “a,” “an,” and “the” include plural reference unless the context clearly dictates otherwise.

As used herein, the term “nucleic acid molecule” is intended to encompass any DNA molecule of interest, including but not limited to a naturally occurring gene, a synthetic gene, or a portion of a naturally occurring or synthetic gene. According to embodiments of the present invention, nucleic acid molecules can be obtained by any method known in the art in view of the present disclosure including, but not limited to, enzymatic methods, such as polymerase chain reaction (PCR) amplification, and chemical methods, such as de novo synthesis on-bead, on-chip gene synthesis, and DNA microarray synthesis.

As used herein, the term “gene” refers to a segment of DNA involved in producing a functional RNA. A gene can include the coding region, non-coding regions preceding (“5′UTR”) and following (“3′UTR”) the coding region, alone or in combination. The functional RNA can be an mRNA that is translated into a peptide, polypeptide, or protein. The functional RNA can also be a non-coding RNA that is not translated into a protein species, but has a physiological function otherwise. Examples of the non-coding RNA include, but are not limited to, a transfer RNA (tRNA), a ribosomal RNA (rRNA), a micro RNA, a ribozyme, etc. A “gene” can include intervening non-coding sequences (“introns”) between individual coding segments (“exons”). A “coding region” or “coding sequence” refers to the portion of a gene that is transcribed into an mRNA, which is translated into a polypeptide and the start and stop signals for the translation of the corresponding polypeptide via triplet-base codons. A “coding region” or “coding sequence” also refers to the portion of a gene that is transcribed into a non-coding but functional RNA.

As used herein, the term “heteroduplex” refers a double stranded nucleic acid molecule having a target sequence comprising one or more sequence errors in one strand and a complementary sequence of the target sequence free of the one or more sequence errors in the other strand. The heteroduplex comprises one or more mismatch sites resulting from the one or more sequence errors.

According to embodiments of the present invention, a heteroduplex can contain multiple mismatch sites, all of which can be corrected simultaneously by a method for correcting a sequence error as described herein. According to embodiments of the present invention, a mismatch site on the heteroduplex can comprise a substitution, a deletion, or an insertion. The mismatch can comprise anywhere from 1 to at least 12 nucleotides, such as a mismatch of at least 1, 2, 3, 4, 5, 6, 7, 8, 9, 10, 11, or 12 nucleotides.

As used herein, a “sequence error” or “error” refers to any change in the nucleotide sequence of a nucleic acid molecule that is different from the desired target sequence for the nucleic acid molecule. The sequence error can be a substitution, insertion, or deletion of in the sequence. Preferably, the error is a substitution, insertion, or deletion, of 1-12 nucleotides.

As used herein, a “CEL endonuclease” refers to a member of a family of DNA mismatch-specific endonucleases originally isolated from plant, which is an ortholog of S1 nuclease, prefers double-stranded mismatched DNA substrates, and can cut a mismatch site in a heteroduplex efficiently at neutral pH. CEL endonucleases have been isolated from various plants, such as celery (Yang et al, Biochemistry 39:3533-3541 (2000), Oleykowski et al, Nucleic Acids Res. 26:4597-4602 (1998)) and spinach (Pimkin et al., BMC Biotechnology 2007, 7:29).

CEL endonuclease is not inhibited by high GC content, and can cut mismatch-containing heteroduplexes efficiently whether the mismatches are base substitutions, insertions or deletions anywhere from 1 to at least 12 nucleotides. CEL endonuclease is able to act efficiently on molecules with multiple mismatches, even with only five nucleotides between mismatches. Additionally, it can handle substrates anywhere from 40 by to approximately 30 kb. Its broad substrate specificity and low non-specific activity have made CEL nuclease one of the best tools for mismatch detection (Yang et al, Biochemistry 39:3533-3541 (2000), Oleykowski et al, Nucleic Acids Res. 26:4597-4602 (1998), Kulinski et al, Biotechniques 29:44 (2000), Yeung et al, Biotechniques 38:749-758 (2005), Qiu et al, Biotechniques 36:702-707 (2004)).

As used herein, the term “overlap extension polymerase chain reaction” or “OE-PCR” refers to an in vitro technique to join together two or more nucleic acid fragments that contain complementary sequences at the ends. When the fragments are mixed, denatured and reannealed, the strands having the matching complementary sequences at their ends overlap and act as primers for each other. Extension of this overlap by DNA polymerase produces a molecule in which the original sequences are spliced together.

According to embodiments of the present invention, the OE-PCR links and amplifies the cleaved fragments of the heteroduplex into a full-length, mismatch-site free nucleic acid molecule. During overlap-extension PCR, the 3′→5′ exonuclease activity of the proof-reading DNA polymerase chews away any 3′ overhangs, which can contain mismatched bases, insertions or deletions, produced by cleavage of the mismatch site by the CEL endonuclease. The error-free fragments are extended and amplified into full-length nucleic acid molecules or gene constructs by the proof-reading DNA polymerase. Intact and error-free nucleic acid duplexes can also be amplified by the overlap extension PCR amplification in the presence of a pair of primers encompassing both ends of the nucleic acid molecule.

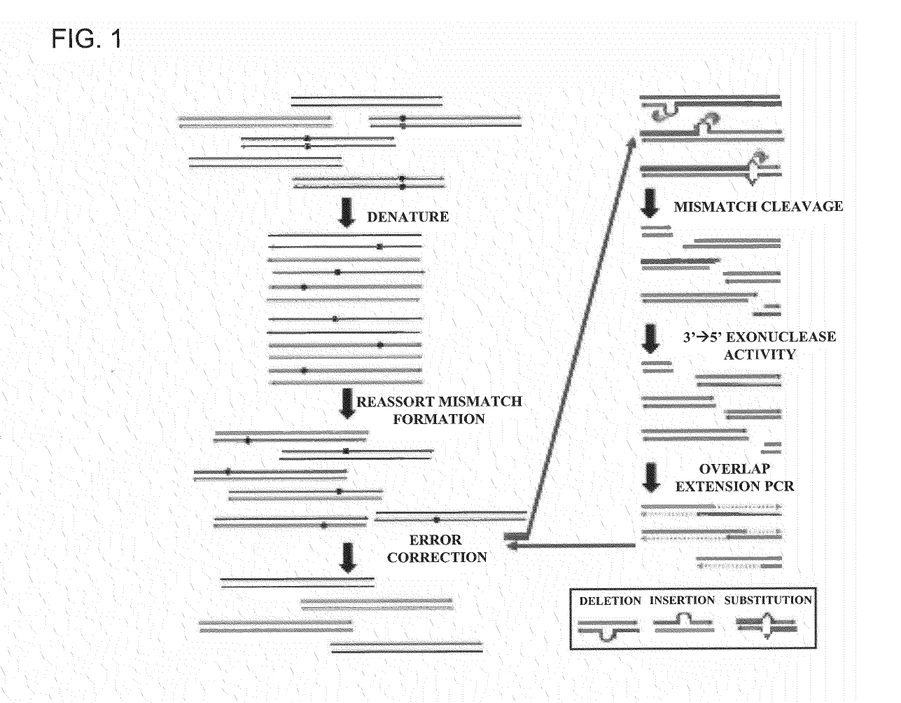

A method for removing one or more sequence errors in a plurality of nucleic acid molecules according to the invention is schematically depicted in FIG. 1. A method of the present invention can be used to correct sequence errors in nucleic acid molecules that are introduced enzymatically, for example during PCR amplification, chemically, for example during DNA microarray synthesis or on-chip gene synthesis, or biologically, for example during replication in a cell.

In a preferred embodiment, the plurality of nucleic acid molecules are synthesized and assembled from a microchip. In a particularly preferred embodiment, the plurality of nucleic acid molecules are genes, wherein the genes are synthesized and assembled from a microchip. The plurality of nucleic acid molecules can be purified, e.g., by agarose gel electrophoresis and extracted, prior to being used in the error correction method according to the present invention. Preferably, the plurality of nucleic acid molecules have the same target sequence except for the sequence errors.

According to embodiments of the present invention, a method for removing one or more sequences errors in a plurality of nucleic acid molecules first comprises forming one or more heteroduplexes from the plurality of nucleic acid molecules, wherein the heteroduplex comprises one or more mismatch sites resulting from the error (FIG. 1, left panel). A heteroduplex can be formed by heating, to denature double-stranded nucleic acid molecules into single-stranded nucleic acid molecules, and subsequently cooling the plurality of nucleic acid molecules. The step of cooling promotes re-annealing of the denatured single-stranded nucleic acid molecules into double-stranded nucleic acid molecules, such that error-free single-stranded nucleic acid molecules can pair with error-containing single-stranded nucleic acid molecules, creating a heteroduplex comprising one or more mismatch-sites.

According to embodiments of the present invention, a plurality of nucleic acid molecules is heated to a temperature that is above the melting temperature of the nucleic acid molecule. One of ordinary skill in the art will be able to readily determine the melting temperature of a nucleic acid molecule. Preferably, the nucleic acid molecules are heated at 94-98° C. for 10 sec to 10 min. For example, the nucleic acid molecules can be heated to a temperature of 95° C. for 10 min. The nucleic acid molecules are then cooled, and preferably slow-cooled, to a temperature between about 15° C. to about 30° C., and preferably to a room temperature of about 25° C. An illustrative and non-limiting example of a procedure of heating and cooling nucleic acid molecules to obtain heteroduplexes according to the invention comprises heating the nucleic acid molecules to 95° C. for 10 min, cooling to 85° C. at 2° C./sec and holding at 85° C. for 1 min, and then cooling to 25° C. at a rate of 0.3° C., holding for 1 min at every 10° C. interval. The appropriate buffer conditions that can be used for heating and slow-cooling the nucleic acid molecules can be chosen based upon the target sequence, e.g., the length, GC content, melting point, etc., as well as the enzymes to be used in subsequent reactions.

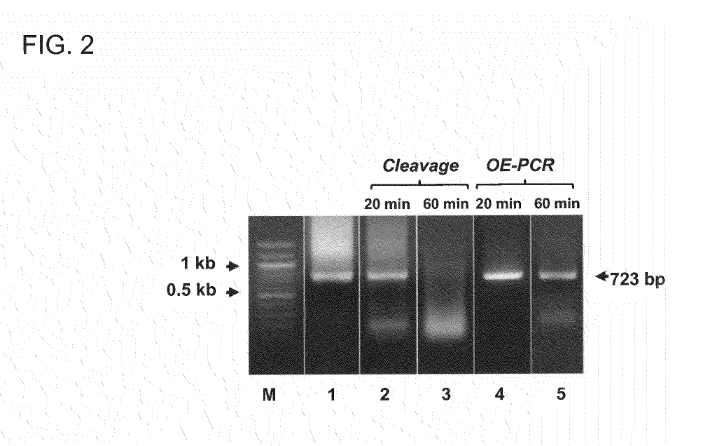

According to embodiments of the present invention, the one or more heteroduplexes obtained from heating and slow cooling the nucleic acid molecules are then contacted with a CEL endonuclease, preferably also a DNA ligase, under conditions that allow for the CEL endonuclease to cleave the one or more heteroduplexes at the mismatch sites, producing cleaved fragments of the nucleic acid molecules (FIG. 1, right panel; FIG. 2, lanes 2 and 3). DNA ligase functions as an enhancer of the CEL endonuclease cleavage reaction (Yeung et al, Biotechniques 38:749-758 (2005); Qiu et al, Biotechniques 36:702-707 (2004); Quan et al, Nat. Biotechnol. 29:449-452 (2011)). Examples of CEL endonucleases that can be used in the present invention include, but are not limited to, CEL I and CEL II endonucleases originally isolated from celery.

According to a preferred embodiment of the present invention, the CEL endonuclease is CEL II endonuclease from celery. As used herein, the term “CEL II endonuclease from celery” refers to a CEL endonuclease originally isolated from celery. It has cleaves with high specificity at the 3′ side of any mismatch site in both DNA strands, including all base substitutions and insertions/deletions up to at least 12 nucleotides (Qiu et al. Qiu et al, Biotechniques 36:702-707 (2004). A CEL II endonuclease from celery is commercially available as Surveyor® endonuclease from Transgenomic as part of the Surveyor Mutation Detection Kit, but can also be produced/purified using methods known in the art.

It is discovered in the present invention that, compared with other known mismatch-specific binding proteins or endonucleases, CEL II endonuclease from celery has the broadest substrate specificity toward all types of mismatches, yielding superior results than previous methods in robust and effective removal of all error types, especially insertions and deletions.

According to embodiments of the present invention, the CEL endonuclease cleavage reaction of the one or more heteroduplexes is performed at a temperature of 35-55° C., and preferably at a temperature of 42° C. The CEL endonuclease cleavage reaction is incubated for 10-60 min, and preferably for about 20 min.

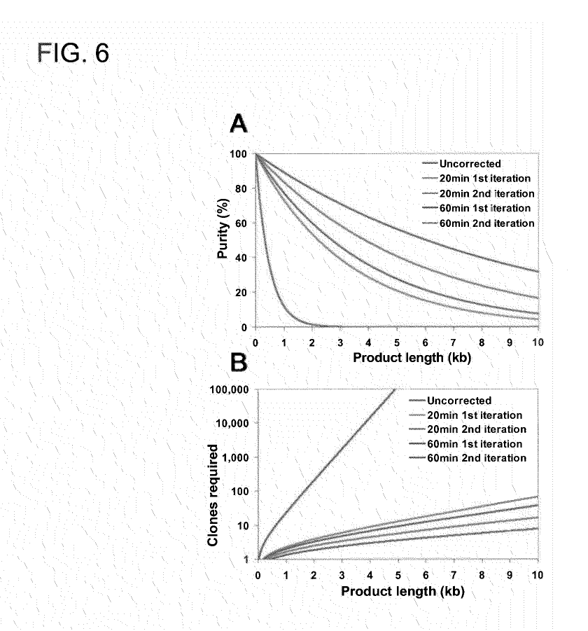

In view of the present disclosure, one of ordinary skill in the art will recognize that the incubation time of the CEL endonuclease cleavage reaction can depend upon various factors, including the number of errors in the heteroduplex, the length of the heteroduplex, and the source of the nucleic acid molecules (i.e. enzymatic or chemical synthesis), and will be able to readily adjust the time and temperature of the CEL endonuclease cleavage reaction accordingly. For example, longer nucleic acid molecules obtained from chemical synthesis are likely to contain more sequence errors than shorter chemically synthesized nucleic acid molecules (see FIG. 6A). Therefore, heteroduplexes obtained from longer nucleic acid molecules can be incubated for a longer period of time with a CEL endonuclease to ensure that the CEL endonuclease efficiently cleaves all the heteroduplexes at each mismatch site, as compared to the incubation time required for heteroduplexes obtained from shorter nucleic acid molecules containing fewer mismatch sites.

In a preferred embodiment. CEL endonuclease cleavage is performed at a temperature of 42° C. for 20 min.

CEL endonuclease cleaves each heteroduplex 3′ of the mismatch site, producing fragments comprising 3′ overhangs, wherein the 3′ overhang comprises the mismatch site (Yeung et al., Biotechniques 38:749-758 (2005)). Error-free nucleic acid molecules remain intact and are not affected by CEL endonuclease treatment.

According to embodiments of the present invention, the cleaved fragments of nucleic acid molecules comprising 3′ overhangs where mismatch bases are located are removed by the 3′→5′ exonuclease activity of a DNA polymerase used in the OE-PCR amplification. Examples of DNA polymerases having 3′→5′ exonuclease activity that can be used with the present invention include, but are not limited to Phusion polymerase, platinum Taq DNA polymerase High Fidelity (Invitrogen), Pfu DNA polymerase, etc. In a preferred embodiment, the DNA polymerase is Phusion polymerase.

As used herein, the term “Phusion polymerase” refers to thermal stable DNA polymerase that contains a Pyrococcus-like enzyme fused with a processivity-enhancing domain, resulting in increased fidelity and speed, e.g., with an error rate >50-fold lower than that of Tag DNA Polymerase and 6-fold lower than that of Pyrococcus furiosus DNA Polymerase. It possesses 5′→3′ polymerase activity, 3′→5′ exonuclease activity and will generate blunt-ended products. An example of Phusion polymerase is Phusion® High-Fidelity DNA Polymerase (New England Biolabs).

During the OE-PCR amplification, the 3′→5′ exonuclease activity of the DNA polymerase chews away any 3′ overhangs, which can contain base substitutions, insertions, or deletions, produced by cleavage of the mismatch site by the CEL endonuclease. The error-free fragments are then extended and amplified into full-length nucleic acid molecules free of the errors (FIG. 1, FIG. 2 lanes 4 and 5). Intact and error-free nucleic acid duplexes can also be amplified by the overlap extension PCR amplification. A pair of end primers encompassing both ends of the target sequence are used for the OE-PCR amplification.

In view of the present disclosure, one of ordinary skill in the art will be able to readily determine the appropriate reaction conditions for the OE-PCR amplification reaction, including number of cycles, denaturation temperature, annealing temperature, elongation temperature, etc. An illustrative and non-limiting example of a procedure for performing the OE-PCR amplification reaction according to the invention comprises initial denaturation at 98° C. for 30 sec, followed by 30 cycles of denaturation at 98° C. for 10 sec, annealing at 60° C. sec, and elongation at 72° C. for 30 sec/kb, finishing with a final elongation at 72° C. for 5 min.

The efficiency of a method for removing one or more errors in nucleic acid molecules according to the invention can also be affected by the amount of heteroduplex in the CEL endonuclease cleavage reaction, and the concentration of CEL endonuclease, as well as the incubation temperature and length of the reaction. According to embodiments of the present invention, the concentration of reannealed nucleic acid molecules which comprise the one or more heteroduplexes can be about 40 ng/μL to about 50 ng/μL. The concentration of the CEL endonuclease can be about 2.5-10 ng/μl.

According to embodiments of the present invention, a method for removing one or more errors from nucleic acid molecules can comprise one iteration of (1) forming one or more heteroduplexes, (2) contacting the heteroduplexes with a CEL endonuclease and a DNA ligase to obtain cleaved fragments of the nucleic acid molecules, and (3) performing an OE-PCR amplification, all as described above, or a method can comprise multiple iterations of repeating steps (1)-(3), such as, for 2, 3, 4, or 5 or more iterations. When more than one iteration of removing errors is performed, the nucleic acid molecules obtained from the OE-PCR amplification step in the previous iteration are used for the subsequent iteration. The optimal number of iterations can depend upon a variety of factors such as the length of the nucleic acid molecule, the number of mismatch sites, etc. For example, shorter nucleic acid molecules may require only one iteration, whereas longer nucleic acid molecules may require two or more iterations to obtain an error free population of nucleic acid molecules (FIG. 6A)

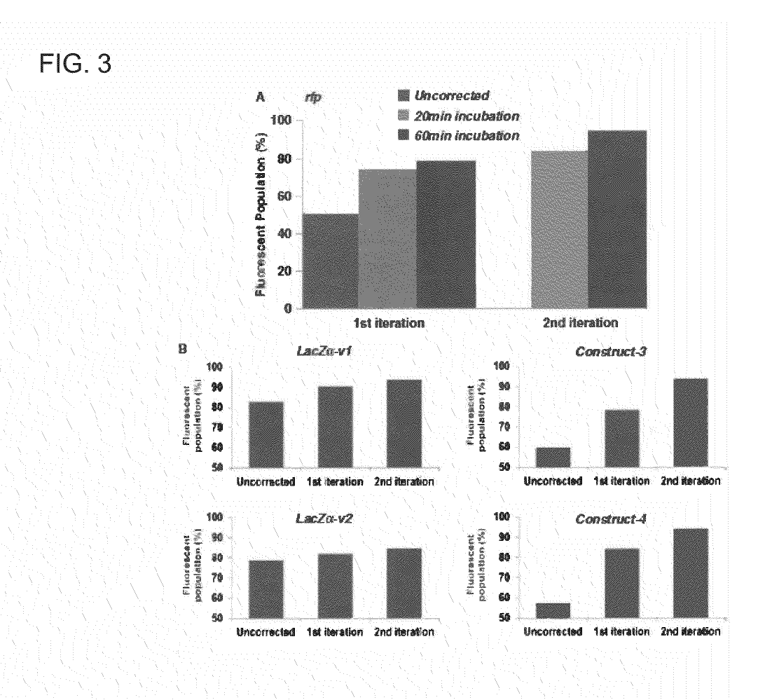

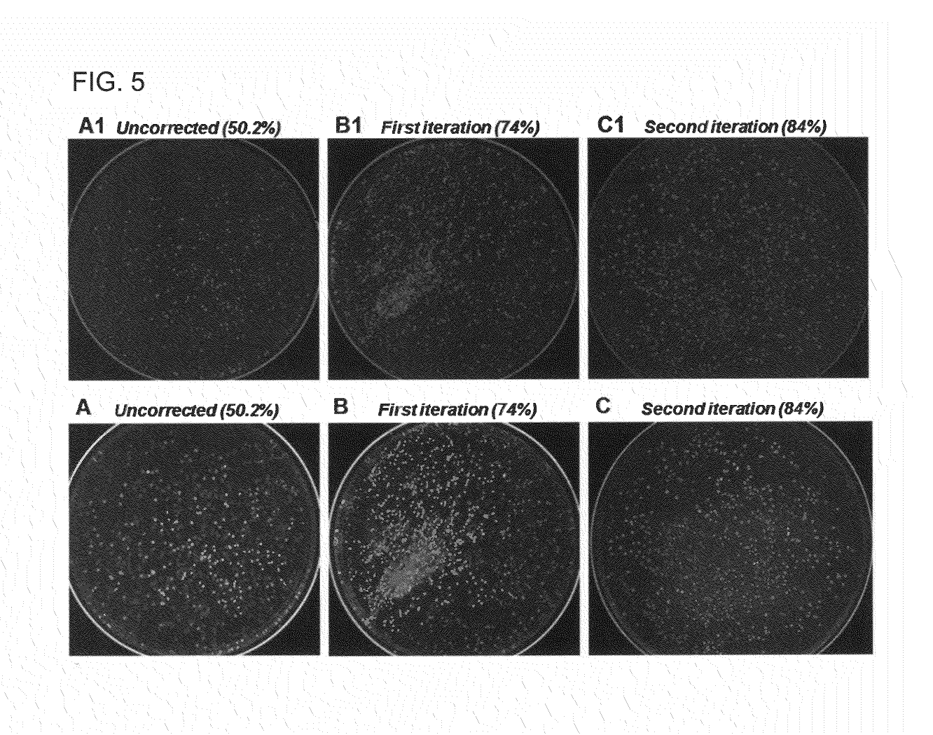

The error-free nucleic acid molecules obtained from OE-PCR amplification can be used in a variety of downstream applications in the fields of biochemistry, molecular biology, and biotechnology, such as, for example, protein expression, cloning, or for the assembly of longer gene products. The nucleic acid molecules can also be operably linked to a reporter gene sequence, such as the gene sequence for red fluorescent protein (RFP), green fluorescent protein (GFP), or lacZ, and transformed into a host cell to evaluate how effectively errors were removed from the nucleic acid molecules, or to determine the optimal number of iterations for obtaining an error-free population of nucleic acid molecules (FIGS. 3, 5). Conventional techniques that are well known to one of ordinary skill in the art can be used for producing such reporter gene constructs, such as for example, circular polymerase extension cloning (CPEC) (Quan and Tian, PloS ONE 4:e6441 (2009)).

According to embodiments of the present invention, removing one or more errors from a nucleic acid molecule according to a method of the invention provides a population of nucleic acid molecules, wherein a larger proportion of the nucleic acid molecules comprise the correct sequence. In addition, performing more than one iteration of removing errors from nucleic acid molecules according to a method of the present invention can increase the probability of obtaining a clone comprising the correct sequence of the nucleic acid molecule, or of obtaining an error-free population of nucleic acid molecules (FIGS. 3, 5, 6B, and 7). For example, using a method of the present invention, the error frequency in nucleic acid molecules can be reduced by >16-fold after two iterations of error correction. This is a significant improvement compared to the about 3.5-fold or 2.9-fold reduction observed by enzymatic correction with T4 endonuclease VII or E. coli endonuclease V, respectively (Fuhrmann et al., Nucleic Acids Res. 2005).

The following examples are to further illustrate the nature of the invention. It should be understood that the following examples do not limit the invention and that the scope of the invention is determined by the appended claims.

### EXAMPLES

The following abbreviations are used in the examples unless stated otherwise:
Polymerase chain reaction (PCR)
Overlap extension polymerase chain reaction (OE-PCR)
Green fluorescent protein (GFP)
Red fluorescent protein (RFP)
Transcription factor (TF)
Error correction reacton (ECR)

### Example 1

### Error Correction Reaction According to a Method of the Present Invention

Provided below is a detailed characterization of the molecular mechanism of the Surveyor-based sequence error correction reaction, referred to as an error correction reaction (ECR), and the development of an optimized ECR protocol which further reduced the error rate down to 1 error in 8,700 base pairs. The nucleic acid molecules used were obtained from on-chip gene synthesis and assembly, as described below.

Reagents.

Chemicals were purchased either from Sigma-Aldrich or VWR. Enzymes were from New England Biolabs. The Surveyor nuclease was purchased from Transgenomic as part of the Surveyor Mutation Detection Kit. GC5 chemical competent cells were purchased from Invitrogen.

Oligonucleotide synthesis and on-chip gene assembly. Oligonucleotides were synthesized on a plastic chip using a custom-made inkjet DNA microarray synthesizer (Saaem et al, ACS Applied Materials & Interfaces 2:491-497 (2010)). Gene-construction oligos were designed to be 60-nucleotides long with overlapping regions of similar melting temperatures (Tm=65±2° C.). The exact oligonucleotides synthesized were those having SEQ ID NOS: 23-40. On-chip oligo amplification and gene assembly using combined nicking strand displacement and polymerase cycle assembly reaction was performed as described with minor modifications (Quan et al, Nat. Biotechnol. 29:449-452 (2011)). Briefly, an 8-well incubation adapter (Sigma-Aldrich) was fitted onto the cyclic olefin polymer chips (COC) so that each well contained a synthesized oligo array. The wells were filled with an strand-displacement amplification and polymerase cycling assembly reaction cocktail composed of 0.4 mM dNTP, 0.2 mg/ml BSA, Nt.Bst NBI, Bst large fragment, and Phusion polymerase in an optimized Thermopol II buffer. The chips with sealed chambers were placed on the in situ slide-adapter of a Mastercycler Gradient thermocycler (Eppendorf) to perform combined strand-displacement amplification and polymerase cycling assembly reactions. Strand-displacement amplification involved incubation at 50° C. for 2 hours followed by 80° C. for 20 min; the polymerase cycling assembly reaction involved an initial denaturation at 98° C. for 30 sec, followed by 40 cycles of denaturation at 98° C. for 7 sec, annealing at 60° C. for 60 sec, and elongation at 72° C. for 15 sec/kb, and finished with an extended elongation step at 72° C. for 5 mm.

After the combined strand-displacement amplification and polymerase cycling assembly reactions, 1-2 μl of the reaction from each chamber was used for PCR amplification with Phusion polymerase and end primers having SEQ ID NOS: 41-43. End primers were employed at a concentration of 0.5 μM. The PCR reaction involved an initial denaturation at 98° C. for 30 sec, followed by 30 cycles of denaturation at 98° C. for 10 sec, annealing at 60° C. for 60 sec, and elongation at 72° C. for 30 sec/kb, and finished with a final elongation at 72° C. for 5 min.

Error Correction Reaction of Assembled Genes.

Once PCR amplification of the on-chip assembled nucleic acid molecule was completed, the gene products were purified by agarose gel electrophoresis and extracted to yield a concentration of >100 ng/μL (measured using a Nanodrop analyzer). These PCR products were then diluted with either 1× Tag buffer or 1× Phusion HF buffer to yield a final concentration of 50 ng/μL. The resulting mixture was then melted by heating at 95° C. for 10 minutes, cooled to 85° C. at 2° C./s and held for 1 min. It was then cooled down to 25° C. at a rate of 0.3° C./s, holding for 1 min at every 10° C. interval.

For ECR using a 20 min Surveyor cleavage incubation, 4 μl (200 ng) of the re-annealed nucleic acid molecule product was mixed with 0.5 μl of Surveyor nuclease and 0.5 μl enhancer (which is known to be DNA ligase in nature and enhances the reaction (Yeung et al, Biotechniques 38:749-758 (2005), Qiu et al, Biotechniques 36:702-707 (2004), Quan et al, Nat. Biotechnol. 29:449-452 (2011)) and incubated at 42° C. for 20 min. 2 μl of the reaction mixture was used for subsequent overlap extension PCR (OE-PCR) using the same reaction conditions as the PCR above, and end primers having SEQ ID NOS: 41-42. The OE-PCR product was cloned and sequenced to serve as the result from the first iteration of error correction. For the second iteration of error correction, the OE-PCR product band was diluted to 50 ng/μL using 1× Taq buffer and re-annealed as before. Similar to the first iteration, a 5 μL reaction consisting of 4 μL re-annealed product, 0.5 μL of Surveyor nuclease and 0.5 μL enhancer was incubated at 42° C. for 20 min. 2 μL of the product was subjected to overlap extension PCR amplification, cloned and sequenced to serve as the result from the second iteration of error correction.

For ECR using a 60 min Surveyor cleavage incubation, 8 μl of the re-annealed nucleic acid molecule product in 1× Phusion buffer (final DNA concentration of 50 ng/μl) was added to 2 μl of Surveyor nuclease and 1 μl enhancer to yield a total of 11 μl that was then incubated at 42° C. for 60 min. 2 μl of the reaction mixture was then subjected to overlap extension PCR amplification, and the resulting PCR product was cloned and sequenced to serve as the result from the first iteration of error correction. For the second iteration, the product from the first iteration was diluted to 50 ng/μl using 1× Phusion buffer and re-annealed as before. Similar to the first iteration, an 11 μl reaction consisting of 8 μl of re-annealed product, 2 μl of Surveyor nuclease and 1 μl of enhancer was incubated at 42° C. for 60 min. 2 μl of the product was used for overlap extension PCR amplification and the PCR product was cloned and sequenced to serve as the result from the second iteration of error correction.

Cloning, Sequencing, and Functional Analysis of Synthetic Genes.

Synthetic gene products, before or after ECR, were cloned into pAcGFP I vector using circular polymerase extension method (CPEC) (Quan and Tian, Nature Protocols 6:242-251 (2011), Quan and Tian, PLoS One 4:e6441 (2009)). Briefly, 250 ng of the linear vector was mixed with the synthetic gene products at 1:2 molar ratios in a 25 μl CPEC reaction using Phusion polymerase. The reaction involved 10 cycles of denaturation at 98° C. for 10 seconds, annealing at 55-60° C. for 30 seconds and extension at 72° C. for 15 seconds, and finished with an extended elongation step at 72° C. for 5 min.

2 μl of the cloning product was transformed into GC5 chemically competent cells (Invitrogen) according to the manufacturer's instructions. Cells were grown on agar plates with 100 μg/ml carbenicillin for approximately 16 hours and then kept at room temperature for 48 hours before being imaged in an Alphalmage gel documentation system. The percentage of fluorescent colonies was automatically determined using CellC program (http://sites.google.com/site/cellcsoftware/download). The results were verified by thresholding the UV images using Adobe Photoshop and counting using ImageJ. Sequence analysis was done by extracting plasmids from randomly selected colonies using a miniprep kit (Qiagen), and sequencing of the plasmids was performed at the Duke University Sequencing Facility.

Results

General Design of the Error Correction Reaction using Surveyor Nuclease

This study relates to the development of a simple and convenient method to effectively remove errors from synthetic genes. The general strategy of using the Surveyor endonuclease to correct errors in synthetic genes is illustrated in FIG. 1. After gene synthesis, the products are denatured and re-annealed to form mismatch-containing heteroduplexes (left panel). The subsequent error correction reaction (ECR, right panel) involves incubation of the re-annealed product with the Surveyor nuclease, followed by overlap extension PCR (OE-PCR) using a proofreading DNA polymerase. The 3′→5′ exonuclease activity of the DNA polymerase removes 3′ overhangs that contain the mismatch base(s) and allows overlap extension to proceed efficiently.

Mismatch structures formed at the deletion, insertion and substitution sites in the heteroduplexes are recognized by the Surveyor mismatch-specific endonuclease, which cuts each strand at the phosphodiester bond at the 3′ side of the mismatch site (Yeung et al, Biotechniques 38:749-758 (2005)). During the subsequent OE-PCR reaction, the 3′→5′ exonuclease activity of the proof-reading DNA polymerase chews away any 3′ overhangs that contain the mismatch base(s) (substitutions and insertions). Finally, the error-free fragments are extended and amplified into full-length gene constructs by the DNA polymerase.

Determination of Error Frequency of On-Chip Gene Synthesis

Integrating oligo synthesis with gene assembly on a microchip can significantly reduce synthesis cost and increase throughput. As described above, DNA microarrays were synthesized using a custom inkjet DNA synthesizer and a combined nSDA-PCA reaction was used for on-chip oligo amplification and gene assembly. To determine error frequency of on-chip gene synthesis without error correction, red fluorescent protein (RFP) was chosen as a test gene for convenient screening of functionally correct genes, which served as a good approximation of sequence correct genes. After the combined strand-displacement amplification and polymerase cycling amplification reactions, the 723-bp rfp construct was amplified by PCR (FIG. 2, lane 1) and inserted into a modified pAcGFP1 expression vector using the CPEC cloning method as described above. After transformation into bacteria, the colonies produced were either non-fluorescent, dimly or brightly fluorescent. A rough approximation of synthesis quality without error correction could be made using colony counts on agar plates. Using automated colony counting, it was found that 50.2% of the rfp colonies formed from uncorrected product fluoresced brightly (FIG. 3A, 5).

DNA sequencing was performed on 42 randomly picked rfp colonies from both directions. Random clones of synthetic genes before (Without ECR) or after one or two ECR iterations (ECR1, ECR2) were sequenced in both directions. Surveyor incubation time (20 min or 60 min) was indicated. The occurrence of different types of errors was counted. The results are shown below in Table 1. The sequencing results indicate an error rate of approximately 1.9/kb Deletions were found to be the dominant form of errors (75.4%), which was similar to column DNA synthesis where monomers are not successfully added to all of the growing polymer chains.

TABLE 1
Error analysis of synthetic gene sequences before and after ECR with
Surveyor nuclease.

| | Uncor- | 20 min | 20 min | 60 min | 60 min |
|---|---|---|---|---|---|
| Error Type | rected | #1 | #2 | #1 | #2 |
| Deletion | | | | | |
| Single-base | 30 | 2 | 0 | 0 | 0 |
| deletion | | | | | |
| Multiple- | 13 | 1 | 0 | 0 | 0 |
| base | | | | | |
| deletion | | | | | |
| Insertion | | | | | |
| Single-base | 3 | 0 | 0 | 0 | 0 |
| insertion | | | | | |
| Multiple- | 1 | 0 | 0 | 0 | 0 |
| base | | | | | |
| insertion | | | | | |
| Substitution | | | | | |
| Transition | | | | | |
| G/C to A/T | 3 | 2 | 1 | 3 | 2 |
| A/T to G/C | 3 | 4 | 2 | 0 | 1 |
| Transversion | | | | | |
| G/C to C/G | 0 | 0 | 0 | 2 | 0 |
| G/C to T/A | 1 | 1 | 1 | 4 | 1 |
| A/T to C/G | 2 | 0 | 0 | 1 | 2 |
| A/T to T/A | 1 | 0 | 1 | 1 | 0 |
| Total errors | 57 | 10 | 5 | 11 | 6 |
| Bases | 29958 | 31866 | 27798 | 42714 | 52206 |
| sequenced | | | | | |
| Error | 1.90 | 0.31 | 0.18 | 0.26 | 0.11 |
| frequency | | | | | |
| (errors per | | | | | |
| kb) | | | | | |
| Error | 526 | 3187 | 5560 | 3883 | 8701 |
| frequency | | | | | |
| (bases per | | | | | |
| error) | | | | | |

Error Correction Reaction with Surveyor Nuclease

Surveyor nuclease has typically been used for mutation detection. A strategy was devised for using it for eliminating errors in synthetic genes, as shown in FIG. 1. To determine the optimal reaction conditions of using Surveyor nuclease for error correction, reaction parameters were systematically varied, such as reagent amount, buffer composition, incubation time, temperature, and number of iterations.

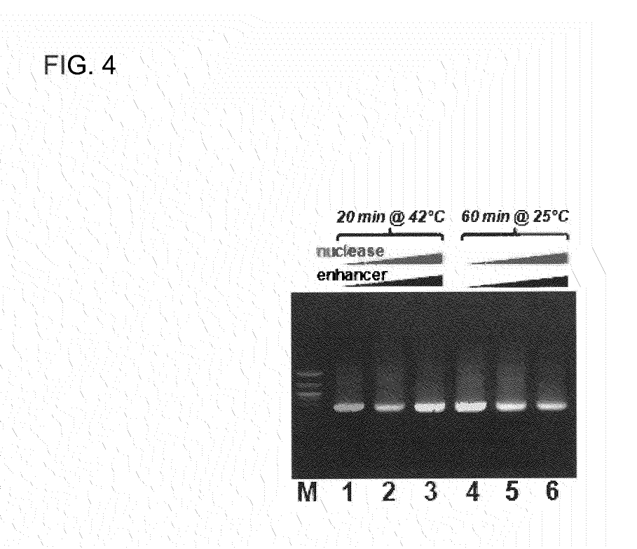

In the first set of experiments, varying amounts of the Surveyor nuclease reagents, including the enzyme and the enhancer were tested. 0.5, 1 and 2 μl of Surveyor nuclease reagents were mixed with 200 ng of re-annealed synthetic rfp product. Incubations were performed either at 42° C. for 20 min or 25° C. for 60 min. After overlap extension PCR amplification, products from all variations were run on an agarose gel (FIG. 4). All bands on the gel appeared to be similar, indicating little difference with increased enzyme concentration.

Depending on the length and sequence quality of the synthetic gene products, after re-annealing and incubation with the Surveyor nuclease, the amount of intact full-length product that can survive the cleavage may be very limited. To assess the extent of cleavage of the on-chip synthesized RFP genes, the re-annealed product was incubated with Surveyor for either 20 or 60 minutes at 42° C. FIG. 2 shows that after 20 min of Surveyor treatment, a fraction of the synthetic genes was cleaved into smaller fragments (lane 2); after 60 min, the majority of the genes were cleaved (lane 3). The results suggested that the cleavage by Surveyor nuclease was relatively efficient. It also suggests that the Surveyor cleavage assay can be used as a quick assessment of the sequence quality of the synthetic products. Following cleavage, overlap extension PCR amplification was able to assemble and extend the fragments back to full-length genes, as shown in FIG. 2 (lanes 4 and 5).

Reduction of Error Frequencies after ECR

Both functional colony counting and DNA sequencing were performed to estimate error frequencies of chip-synthesized genes after ECR with 20-min or 60-min Surveyor treatment. It was reasoned that in one round of ECR, sequences containing errors could form homodimers by chance during annealing and thus escape detection and cleavage. A test was, therefore, made to determine whether an additional round of ECR could eliminate more errors. Two iterations of ECR were performed with both 20 min and 60 min incubations as outlined above. Full-length gene products were cloned and used for functional assays and Sanger sequencing in order to estimate error frequencies.

As shown in FIG. 3A, increasing Surveyor cleavage time and number of iterations led to increases in the number of brightly fluorescent colonies. Using 20-min Surveyor treatment, the fluorescent population increased from 50.2% (untreated) to 74% and 84% in the first and second iteration, respectively. Using 60-min Surveyor treatment resulted in 78.4% and 94% fluorescent colonies after the first and second iteration. Example images showing the fluorescent colonies can be found in FIG. 5.

To investigate the repeatability and robustness of the method, it was applied to the synthesis of four additional gene constructs and its effectiveness was measured using functional or reporter assays. Of the four constructs, two were codon variants of the lacZa gene, the expression of which cause the colony to turn blue in the presence of X-gal. The other two constructs could not be screened by their own functions and, therefore, were fused to the N-terminus of the green fluorescent protein (GFP) (FIG. 3B). Blue or fluorescent colonies indicated that there were no frame shifts or mutations in the gene constructs that could abolish the function or expression of the genes. Therefore, the percentage of positive colonies could be used as an approximate indicator of the quality of the sequences. The results from the four additional constructs showed iterative increase in positive populations after each round of ECR (FIG. 3B). As expected from the model predictions shown in FIG. 6A, the small lacZa genes had a large fluorescent population even before error correction (80% positive) as it had fewer errors to begin with due to their short length (174 bp). In comparison, the longer constructs (#3 & 4) had lower percentages of correct colonies to begin with (500 bp, 55-60% positive) but the effect of ECR was more obvious, reaching >90% positive after two iterations (FIG. 3B).

Results from DNA sequencing analysis of randomly selected colonies correlated with the observations made with the colony counting experiments and revealed more details on the correction efficiency of different types of errors. The results in Table 1 showed that ECR with Surveyor was very efficient in reducing errors arising from deletion and insertion events. Most deletion and insertion type of errors could be eliminated after one round of 60-min treatment or two rounds of 20-min treatment. Surveyor treatment was also effective towards substitutions albeit with reduced efficiency. Substitution types of errors were still present after two rounds of 60-min incubations.

For the purpose of developing the most efficient ECR procedure, data in Table 1 indicated that increasing incubation time from 20 min to 60 min reduced error frequency from 0.31 to 0.26 error/kb (16% reduction); while adding another round reduced error frequency from 0.31 to 0.18 error/kb for 20-min incubations (42% reduction) and from 0.26 to 0.11 error/kb for 60-min incubations (58% reduction). It appeared that adding a second round of ECR was more effective than increasing the Surveyor incubation time with only one round of ECR, although the cumulative effects of more iterations and longer Surveyor incubation was most dramatic.

Following the model predictions of Carr et al (Nucleic Acids Res. 32:e162 (2004)) and Furhmann et al (Nucl. Acids Res. 33(6):e58 (2005)), statistical analysis was performed to better understand the implication of the results. As can be seen in FIG. 6A, the percentage of gene synthesis products that yield error-free clones decreases exponentially with the length of the product synthesized. Employing ECR for error correction (1 error in 8701 bp for two iterations of 60-min ECR, blue line) significantly increases the probability of locating an error-free clone than without error correction (1 error in 526 bp, red line). This means that dramatically fewer clones need to be sequenced (FIG. 6B). For example, as predicated in FIG. 6B, with ECR one will have to screen, on average, only 8-10 clones of a 10 kb treated or a single 1 kb clone in order to locate a correct one. The model prediction correlated well with the sequencing analysis results.

Analyzing sequencing data of 77 random colonies from the second iteration of the 60-min ECR, 72 of the colonies were found to contain the correct rfp gene. The determined error rate of 0.11/kb meant a >16-fold reduction of errors present in the synthetic pool. With such an improvement, larger DNA targets can be conveniently synthesized and corrected within 2-3 hours without resorting to additional cloning or excessive sequencing.

In conclusion, the method described above performs enzymatic error correction on synthetic genes using Surveyor nuclease, which has the broadest substrate specificity towards all types of mismatches as compared to other known mismatch-specific binding proteins or endonucleases. The method utilizes the mismatch-specific endonuclease activity of the Surveyor enzyme to cut heteroduplex sequences at the mismatch sites and uses the exonuclease activity of the proof-reading DNA polymerase to remove the mismatch bases, followed by an OE-PCR reaction to reassemble the cleaved fragments into full-length gene constructs. The results demonstrate that the optimized ECR procedure is robust and effective for all error types, especially insertions and deletions, yielding superior results than previous methods. The ECR method is probably more suitable for long and error-rich synthetic products and can be performed in less time than MutS-based procedures which require gel shift assay and DNA extraction from polyacryalamide gels. Additionally, in comparison to the commercial ErrASE kit (Kosuri et al, Nat. Biotech. 28:1295-1299 (2010)), the ECR reaction mitigates the need for tittering and excessive enzyme usage. Using the protocol developed in the current study, two ECR iterations could be completed in less than 5 hours and reduces the error frequency by >16-fold.

### Example 2

### Error Correction Reaction

Example 1: Synthesis of a nucleic acid molecule on-chip, enzymatic error correction, and screening a library of codon variants.

Oligonucleotide Synthesis on Cyclic Olefin Polymer (COC) Chips.

Oligonucleotide synthesis, amplification and assembly were performed on the same chip in an effort to achieve additional increases in the throughput of nucleic acid molecule synthesis. Chip oligos were synthesized using a custom-made inkjet DNA rnicroarray synthesizer on embossed cyclic olefin copolymer (COC) chips (Ma et al, J. Mater. Chem. 19:7914-7920 (2009); Saaem et al, ACS Applied Materials and Interface 2:491-497 (2010)). Gene construction oligos were designed to be 48 or 60 bases long with a 25-base universal adaptor sequence at the 3′end, which provided a nicking site and anchored the oligonucleotide to the surface of the COC chip. The oligonucleotide sequences synthesized comprised a portion of a gene sequence of red fluorescent protein gene (SEQ ID NOS: 1-18). In the current designs, COC chips were partitioned to form 8 or 30 subarrays of silica thin-film spots 150-μm in diameter and 300-μm in interfeature spacing (center to center). Each chamber, or subarray, in the 30-chamber design could print 361 spots and was used to synthesize only one gene, or gene library up to 0.5-1 kb in length. Multiple spots were used to synthesize one oligonucleotide sequence.

Combined nSDA-PCA Reaction for On-Chip Oligo Amplification and Gene Assembly.

The chambers on the printed COC slides were filled with the nSDA-PCA reaction cocktail containing 0.4 rnM dNTP, 0.2 mg/ml bovine serum albumin (BSA), Nt.BstNBI, Bst large fragment, nSDA primer (SEQ ID NO: 19) and Phusion polymerase in optimized Thermopol II buffer. The slides with sealed chambers were placed on the slide adaptor of a Mastercycler Gradient thermocycler (Eppendorf) and the combined nSDA-PCA reactions were carried out. nSDA involved incubation at 50° C. for 2 h followed by 80° C. for 20 min; the subsequent PCA reaction involved an initial denaturation at 98° C. for 30 s, followed by 40 cycles of denaturation at 98° C. for 7 s, annealing at 60° C. for 60 s, and elongation at 72° c for 15 s/kb, and finished with an extended elongation step at 72° C. for 5 min. The primers used for the PCR amplification are shown in SEQ ID NOS: 20-22.

PCR Amplification of Assembled Nucleic Acid Molecule.

After nSDA-PCA reaction, 1-2 μl of the reaction from each chamber was used for PCR amplification with Phusion polymerase. The PCR reaction involved an initial denaturation at 98° C. for 30 sec, followed by 30 cycles of denaturation at 98° C. for 10 sec, annealing at 60° C. for 60 sec, and elongation at 72° C. for 15 sec/kb, and finished with a final elongation at 72° C. for 5 min. The primers used for the PCR amplification are shown in SEQ ID NOS: 20-21.

Enzymatic Error Correction.

Chip-synthesized genes were diluted in 1×Taq buffer, and were denatured and reannealed by incubating at 95° C. for 2 min before cooling down first to 85° C. at a rate of 2° C. per second and then to 25° C. at a rate of 0.1° C. per second. The reaction (4μl was mixed with 1 μl of the Surveyor nuclease reagents (Transgenomic) and incubated at 42° C. for 20 min. The product (2 μl) was PCR amplified, cloned and sequenced.

Image Analysis of E. coli Colonies to Determine Percent of Fluorescent Colonies before and after ECR.

150-mm LB agar plates were spread evenly with transformed E. coli cells and incubated overnight at 37° C. Raw images were acquired by scanning the plates with a computer-controlled HP Photosmart C7180 Flatbed Scanner. Bacterial colonies were then identified as a set of objects ranging from 2 to 30 pixels in diameter on scanned images. An automatic thresholding method using a mixture of Gaussians was used to identify local maxima (Lamprecht et al, Biotechniques 42:71-75 (2007)). The images were converted to grayscale and pixel intensities were inverted. From the set of pixels located in each colony, ten pixels with the maximum intensities were selected and averaged to give an estimate of colony color intensity.

Results

To reduce gene synthesis errors, a simple yet effective error-correction method was developed using the plant CEL family of mismatch-specific endonucleases, which have been shown to recognize and cleave all types of mismatches arising from base substitutions or from small insertions or deletions. A commercial source of a subtype of the CEL enzymes was the Surveyor nuclease, which has been used primarily for mutation detection (Qiu et al, Biotechniques 36:702-707 (2004)). To use it for error correction, the synthetic genes were first denatured by heat and reannealed, and then treated with Surveyor nuclease to cleave error-containing heteroduplexes at the mismatch sites. The error-free DNA duplexes remained intact and were amplified by overlap-extension PCR.

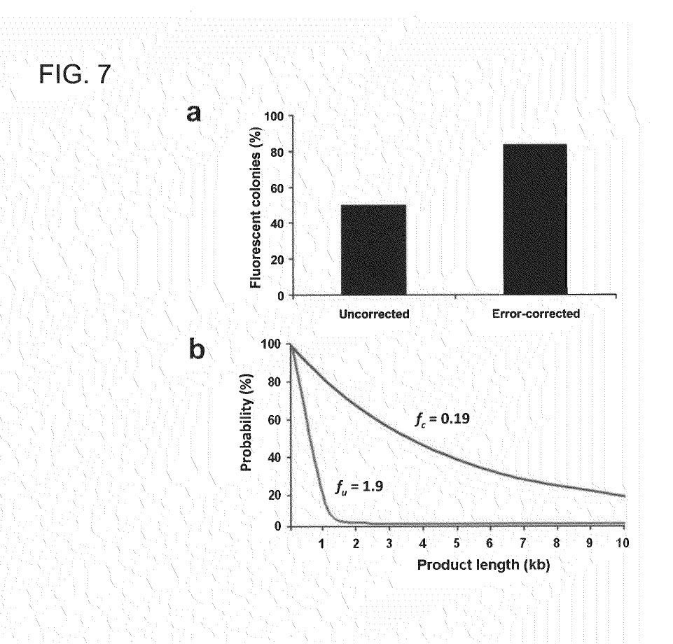

To test the effectiveness of this approach, chip-synthesized genes encoding red fluorescent protein (RFP) were cloned into an expression vector with and without Surveyor nuclease treatment. Sequencing and automated fluorescent colony-counting experiments were performed to determine and compare error frequencies. By Sanger sequencing 470 randomly selected clones, error frequencies of 1/526 by (or 1.9 errors per kb) and 1/5,392 by (or 0.19 errors per kb) were observed before and after Surveyor nuclease treatment, respectively (see Table 2 below). Automated counting of thousands of colonies showed that 50% and 84% of the RFP colonies were fluorescent in untreated and Surveyor nuclease-treated populations (FIG. 7A). The results of the sequencing and the colony counting experiments correlated well according to statistical analysis (FIG. 7B). Another study reported comparable error frequencies using the commercial ErrASE kit (Kosuri et al, Nat. Biotechnol. 28:1295-1299 (2010)).

TABLE 2
Error frequencies as determined by sequencing in chip-synthesized
RFP genes with and without error correction using Surveyor nuclease.
Clones wererandomly selected from each population and sequenced
from both directions.

| | | | | | | Error |
|---|---|---|---|---|---|---|
| | De- | Inser- | Substi- | Total | Bases | Frequency |
| | letions | tions | tutions | Errors | Sequenced | f (per kb) |
| Before | 43 | 4 | 10 | 57 | 29,958 | 1.9 |
| correction | | | | | | |
| Surveyor | 6 | 0 | 48 | 54 | 291,180 | 0.19 |
| correction | | | | | | |

The integration of oligo synthesis and gene assembly on the same microchip facilitates automation and miniaturization, which leads to cost reduction and increases in throughput. On the current chip, each of the 30 chambers was used to synthesize one gene fragment up to 1 kb in length with a 9× redundancy in oligo usage (9 subarray features were used to synthesize one oligo sequence). The estimated cost of chip-oligonucleotide synthesis for this 30kb of sequence was <$0.001/bp of final synthesized sequences, which is one-tenth of the lowest reported cost (Kosuri et al, Nat. Biotechnol. 28:1295-1299 (2010)). Including a step of error correction, the average cost of integrated gene synthesis on a chip is <$0.005/bp of final synthesized gene sequences with an error frequency of <0.2 error/kb. With multiplexing and more advanced chip design, greater throughput and lower costs are potentially achievable.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method for removing one or more sequence errors in a plurality of nucleic acid molecules, the method comprising:
(1) heating and subsequently cooling the plurality of nucleic acid molecules, thereby forming one or more heteroduplexes, wherein the heteroduplex comprises one or more mismatch sites resulting from the sequence errors;
(2) contacting the one or more heteroduplexes with a CEL endonuclease under conditions for effective cleavage of the one or more heteroduplexes at the one or more mismatch sites, thereby obtaining cleaved fragments of the heteroduplexes; and
(3) contacting the cleaved fragments with a DNA polymerase having 3′-5′ exonuclease activity under conditions for an overlap extension polymerase chain reaction amplification, thereby producing a plurality of nucleic acid molecules free of the one or more errors.

2. The method of claim 1, wherein the CEL endonuclease is CEL II endonuclease from celery.

3. The method of claim 1, wherein the sequence error comprises a substitution, deletion or insertion of 1-12 nucleotides.

4. The method of claim 1, further comprising repeating steps (1) to (3).

5. The method of claim 1, wherein the heteroduplex is formed by heating the plurality of nucleic acid molecules at 94-98° C. for 10 sec to 10 min followed by the cooling to room temperature.

6. The method of claim 1, wherein the one or more heteroduplexes are contacted with a CEL endonuclease at 35-55° C. for 10-60 min for effective cleavage of the one or more heteroduplexes at the one or more mismatch sites, thereby obtaining cleaved fragments of the heteroduplexes.

7. The method of claim 1, wherein the one or more heteroduplexes are contacted with a CEL endonuclease at 42° C. for 20 min for effective cleavage of the one or more heteroduplexes at the one or more mismatch sites, thereby obtaining cleaved fragments of the heteroduplexes.

8. The method of claim 1, wherein the DNA polymerase is Phusion polymerase.

9. The method of claim 1, wherein the plurality of nucleic acid molecules are synthesized and assembled from a microchip, and are amplified and purified, prior to step (1).

10. The method of claim 1, wherein a DNA ligase is added in step (2) to enhance the effective cleavage.

11. The method of claim 1, further comprising transforming a cell with the nucleic acid molecules free of the one or more errors obtained in step (3).

12. A kit for removing one or more sequence errors in a plurality of nucleic acid molecules, the kit comprising:
(1) a CEL endonuclease, a DNA ligase and a buffer solution for cleaving a heteroduplex at one or more mismatch sites;
(2) a DNA polymerase having 3′-5′ exonuclease activity and reagents for performing an overlap extension polymerase chain reaction amplification; and
(3) instructions on using the kit for removing one or more sequence errors in a plurality of nucleic acid molecules.

13. The kit of claim 12, wherein the CEL endonuclease is CEL II endonuclease from celery.

14. The kit of claim 12, wherein the DNA polymerase is Phusion polymerase.

15. A method for removing one or more sequence errors in a plurality of nucleic acid molecules, the method comprising:
(1) heating and subsequently cooling the plurality of nucleic acid molecules, thereby forming one or more heteroduplexes, wherein the heteroduplex comprises one or more mismatch sites resulting from the sequence errors;
(2) contacting the one or more heteroduplexes with a CEL II endonuclease from celery and a DNA ligase at 35-55° C. for 10-60 min for effective cleavage of the one or more heteroduplexes at the one or more mismatch sites, thereby obtaining cleaved fragments of the heteroduplexes; and
(3) contacting the cleaved fragments with Phusion polymerase under conditions for an overlap extension polymerase chain reaction amplification, thereby producing a plurality of nucleic acid molecules free of the one or more errors.


SEQUENCE LISTING (43 sequences)

SEQ ID NO: 1 - DNA, 85 bp, Artificial Sequence
  RFP-f1-1
ttcaaatgggaacgtgttatgaacttcgaagacggtggtgttgttaccgttacccaggacgcatgactcgaccatccgatttttt

SEQ ID NO: 2 - DNA, 85 bp, Artificial Sequence
  RFP-f1-2
ttcataacacgttcccatttgaaaccttccgggaaggacagtttcaggtagtccgggatggcatgactcgaccatccgatttttt

SEQ ID NO: 3 - DNA, 85 bp, Artificial Sequence
  RFP-f1-3
ccagtacggttccaaagcttacgttaaacacccggctgacatcccggactacctgaaactgcatgactcgaccatccgatttttt

SEQ ID NO: 4 - DNA, 85 bp, Artificial Sequence
  RFP-f1-4
cgtaagctttggaaccgtactggaactgcggggacaggatgtcccaagcgaacggcagcggcatgactcgaccatccgatttttt

SEQ ID NO: 5 - DNA, 85 bp, Artificial Sequence
  RFP-f1-5
acgaaggtacccagaccgctaaactgaaagttaccaaaggtggtccgctgccgttcgcttgcatgactcgaccatccgatttttt

SEQ ID NO: 6 - DNA, 85 bp, Artificial Sequence
  RFP-f1-6
gcggtctgggtaccttcgtacggacgaccttcaccttcaccttcgatttcgaactcgtgagcatgactcgaccatccgatttttt

SEQ ID NO: 7 - DNA, 85 bp, Artificial Sequence
  RFP-f1-7
catgcgtttcaaagttcgtatggaaggttccgttaacggtcacgagttcgaaatcgaagggcatgactcgaccatccgatttttt

SEQ ID NO: 8 - DNA, 85 bp, Artificial Sequence
  RFP-f1-8
catacgaactttgaaacgcatgaactctttgataacgtcttcggaggaagccatcatagcgcatgactcgaccatccgatttttt

SEQ ID NO: 9 - DNA, 85 bp, Artificial Sequence
  RFP-f1-9
agcctggatgacgttttcatcaaaatttcacacaggaaacagctatgatggcttcctccggcatgactcgaccatccgatttttt

SEQ ID NO: 10 - DNA, 85 bp, Artificial
  RFP-f2-1
cgggaagttggtaccacgcagtttaactttgtagatgaactcaccgtcttgcagggaggagcatgactcgaccatccgatttttt

SEQ ID NO: 11 - DNA, 85 bp, Artificial Sequence
  RFP-f2-2
cgtggtaccaacttcccgtccgacggtccggttatgcagaaaaaaaccatgggttgggaagcatgactcgaccatccgatttttt

SEQ ID NO: 12 - DNA, 85 bp, Artificial Sequence
  RFP-f2-3
tcagagcaccgtcttccgggtacatacgttcggtggaagcttcccaacccatggttttttgcatgactcgaccatccgatttttt

SEQ ID NO: 13 - DNA, 85 bp, Artificial Sequence
  RFP-f2-4
cggaagacggtgctctgaaaggtgaaatcaaaatgcgtctgaaactgaaagacggtggtcgcatgactcgaccatccgatttttt

SEQ ID NO: 14 - DNA, 85 bp, Artificial Sequence
  RFP-f2-5
tttagccatgtaggtggttttaacttcagcgtcgtagtgaccaccgtctttcagtttcaggcatgactcgaccatccgatttttt

SEQ ID NO: 15 - DNA, 85 bp, Artificial Sequence
  RFP-f2-6
gaagttaaaaccacctacatggctaaaaaaccggttcagctgccgggtgcttacaaaaccgcatgactcgaccatccgatttttt

SEQ ID NO: 16 - DNA, 85 bp, Artificial Sequence
  RFP-f2-7
gtgtagtcttcgttgtgggaggtgatgtccagtttgatgtcggttttgtaagcacccggcgcatgactcgaccatccgatttttt

SEQ ID NO: 17 - DNA, 85 bp, Artificial Sequence
  RFP-f2-8
cctcccacaacgaagactacaccatcgttgaacagtacgaacgtgctgaaggtcgtcactgcatgactcgaccatccgatttttt

SEQ ID NO: 18 - DNA, 85 bp, Artificial Sequence
  RFP-f2-9
aaattgagactggaaacgcacggtttcttaagcaccggtggagtgacgaccttcagcacggcatgactcgaccatccgatttttt

SEQ ID NO: 19 - DNA, 19 bp, Artificial Sequence
  nSDA Primer
tcggatggtcgagtcatgc

SEQ ID NO: 20 - DNA, 25 bp, Artificial Sequence
  rfp-f
aatttcacacaggaaacagctatga

SEQ ID NO: 21 - DNA, 19 bp, Artificial Sequence
  rfp-r
agactggaaacgcacggtt

SEQ ID NO: 22 - DNA, 39 bp, Artificial Sequence
  rfp-m
tgttgttaccgttacccaggactcctccctgcaagacgg

SEQ ID NO: 23 - DNA, 85 bp, Artificial Sequence; identical to SEQ ID NO: 1
  rfp-f1-1

SEQ ID NO: 24 - DNA, 85 bp, Artificial Sequence; identical to SEQ ID NO: 2
  rfp-f1-2

SEQ ID NO: 25 - DNA, 85 bp, Artificial Sequence; identical to SEQ ID NO: 3
  rfp-f1-3

SEQ ID NO: 26 - DNA, 85 bp, Artificial Sequence; identical to SEQ ID NO: 4
  rfp-f1-4

SEQ ID NO: 27 - DNA, 85 bp, Artificial Sequence; identical to SEQ ID NO: 5
  rfp-f1-5

SEQ ID NO: 28 - DNA, 85 bp, Artificial Sequence; identical to SEQ ID NO: 6
  rfp-f1-6

SEQ ID NO: 29 - DNA, 85 bp, Artificial Sequence; identical to SEQ ID NO: 7
  rfp-f1-7

SEQ ID NO: 30 - DNA, 85 bp, Artificial Sequence; identical to SEQ ID NO: 8
  rfp-f1-8

SEQ ID NO: 31 - DNA, 85 bp, Artificial Sequence; identical to SEQ ID NO: 9
  rfp-f1-9

SEQ ID NO: 32 - DNA, 85 bp, Artificial Sequence; identical to SEQ ID NO: 10
  rfp-f2-1

SEQ ID NO: 33 - DNA, 85 bp, Artificial Sequence; identical to SEQ ID NO: 11
  rfp-f2-2

SEQ ID NO: 34 - DNA, 85 bp, Artificial Sequence; identical to SEQ ID NO: 12
  rfp-f2-3

SEQ ID NO: 35 - DNA, 85 bp, Artificial Sequence; identical to SEQ ID NO: 13
  rfp-f2-4

SEQ ID NO: 36 - DNA, 85 bp, Artificial Sequence; identical to SEQ ID NO: 14
  rfp-f2-5

SEQ ID NO: 37 - DNA, 85 bp, Artificial Sequence; identical to SEQ ID NO: 15
  rfp-f2-6

SEQ ID NO: 38 - DNA, 85 bp, Artificial Sequence; identical to SEQ ID NO: 16
  rfp-f2-7

SEQ ID NO: 39 - DNA, 85 bp, Artificial Sequence; identical to SEQ ID NO: 17
  rfp-f2-8

SEQ ID NO: 40 - DNA, 85 bp, Artificial Sequence; identical to SEQ ID NO: 18
  rfp-f2-9

SEQ ID NO: 41 - DNA, 25 bp, Artificial Sequence
  rfp-f
aatttcacacaggaaacagctatga

SEQ ID NO: 42 - DNA, 19 bp, Artificial Sequence
  rfp-r
agactggaaacgcacggtt

SEQ ID NO: 43 - DNA, 39 bp, Artificial Sequence; identical to SEQ ID NO: 22
  rfp-m